(19) United States
(12) Patent Application Publication
(10) Pub. No.: US 2004/0010302 A1
(43) Pub. Date: Jan. 15, 2004

(54) Cold pack

(76) Inventors: Kristen Lane von Hoffmann, Montclair, NJ (US); Eric William von Hoffmann, Montclair, NJ (US)

Correspondence Address: OSTROLENK, FABER, GERB & SOFFEN, LLP, 1180 AVENUE OF THE AMERICAS, NEW YORK, NY, 10036-8403, US

(21) Appl. No.: 10/193,778

(22) Filed: Jul. 12, 2002

(51) Int. Cl. A61F 7/00

(52) U.S. Cl. 607/114

(57) ABSTRACT

A cold pack is provided for supplying a cooling temperature to an area to be subjected to a cold treatment, the cold pack including a container having an interior space and a plurality of cooling modules situated in the interior space of the container, in which the cooling modules move at least substantially freely relative to one another, without the need for an initial deforming force, and even if the cold pack is applied to the area to be subjected to a cold treatment immediately after being removed from a cooling source, such as a freezer. When applied, the cold pack promotes the cooling effects of the cold pack and better conforms to the area to be subjected to a cold treatment.

### FIELD OF THE INVENTION

The present invention relates to cold packs, for example, ice packs, used for cooling an area to be subjected to a cold treatment, such as an injured area of a human body.

### BACKGROUND INFORMATION

It is believed that U.S. Pat. No. 4,044,773, which is expressly incorporated herein by reference, refers to a cold therapeutic package, in which water is sealed within an interior of a thin polyurethane bladder. The water is then frozen, resulting in the formation of a thin layer of ice within the bladder. After being exposed to a deforming force, such as a rolling or striking force, the layer of ice is divided into a plurality of small ice particles. These small ice particles may move at least substantially freely relative to one another when the cold therapeutic package is applied to an area to be subjected to a cold treatment, thereby promoting easier application of the cold package to the area.

### SUMMARY OF THE INVENTION

It is an object of the present invention to improve conventional cold packs by providing a cold pack design that promotes the cooling effects of the cold pack and better conforms to an area to be subjected to a cold treatment.

It is another object of the present invention to provide a cold pack, which is better suited to provide a cooling temperature to an area to be subjected to a cold treatment.

It is another object of the present invention to provide a cold pack, which does not require a substantial initial deforming force for subdividing a cooling medium, such as ice, into a plurality of smaller particles.

To achieve these objects, an exemplary cold pack according to the present invention includes a container having an interior space and a plurality of cooling modules situated in the interior space of the container, in which the cooling modules move freely, or at least substantially freely, relative to one another without the need for a substantial initial deforming force.

### BRIEF DESCRIPTION OF THE DRAWINGS

FIG. 1 is a cut-away view showing a first exemplary cold pack according to the present invention.

FIG. 2 is a cut-away view showing further detail of the cooling modules shown in FIG. 1.

FIG. 3 is a diagram showing application of an exemplary cold pack according to the present invention.

### DETAILED DESCRIPTION

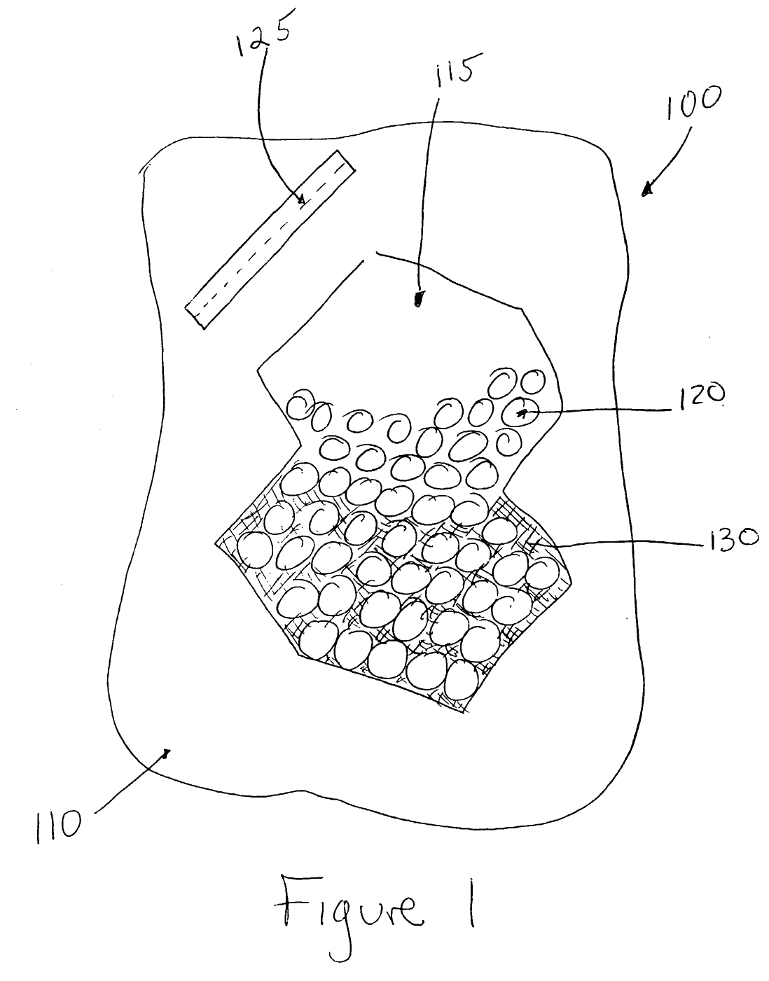

Referring now to FIG. 1, there is seen a cut-away view of a first exemplary cold pack 100 according to the present invention. As shown in FIG. 1, the cold pack 100 includes a container 110 having an interior space 115 and a plurality of cooling modules 120 situated within the interior space 115 of the container 110. For example, the container 110 may include 1055 spherical cooling modules 120, each having a diameter of between about 11/32 inches and 13/32 inches and each weighing, for example, between about 0.4 grams and 0.6 grams. The container 110 may weigh, for example, about 454 grams when filled with the cooling modules 120.

The container 110 may be composed of a strong, durable, rigid, semi-rigid, flexible, malleable, conforming, and/or fabric-like material, for example, rigid plastic, flexible plastic (i.e., a strong and durable plastic bag), canvas, polyurethane, etc.

Furthermore, the container 110 may be designed in any shape, such as oval, square, rectangular, etc. For example, the container 110 may include a substantially rectangular bag dimensioned, for example, about 6½ inches by 9 inches, having a thickness of about 1¼ inches when laid flat.

The container 110 may permanently enclose the cooling modules 120 (i.e., the container 110 may be a permanently sealed unit) or, as shown in FIG. 1, the container 110 may include an access apparatus 125, such as a zipper, velcro, buttons, straps, and/or snaps, operable to permit access to the interior space 115 of the container 110. In this manner, the cooling modules 120 may be replaced if necessary, for example, if at least one of the cooling modules 120 is defective.

The interior space 115 of the container 110 between the cooling modules 120 may remain devoid of any material, or may include, for example, a filling medium 130, as shown in FIG. 1. The filling medium 130 may include, for example, a liquid, a gas, such as air or nitrogen, a gelatinous material, a material resistant to freezing when cooled, such as Freon, salt-water, or anti-freeze, or, for example, a chemical cooling agent that chemically produces a cooling temperature, without the need for an external cooling source, such as a freezer.

It should be noted that the interior space 115 of the container 110 between the cooling modules 120 may be completely filled with the filling medium 130 or, alternatively, may only be partially filled with the filling medium 130. In this manner, the container 110 may permit the filling medium 130 to expand within the container 110 while freezing, without rupturing the container 110.

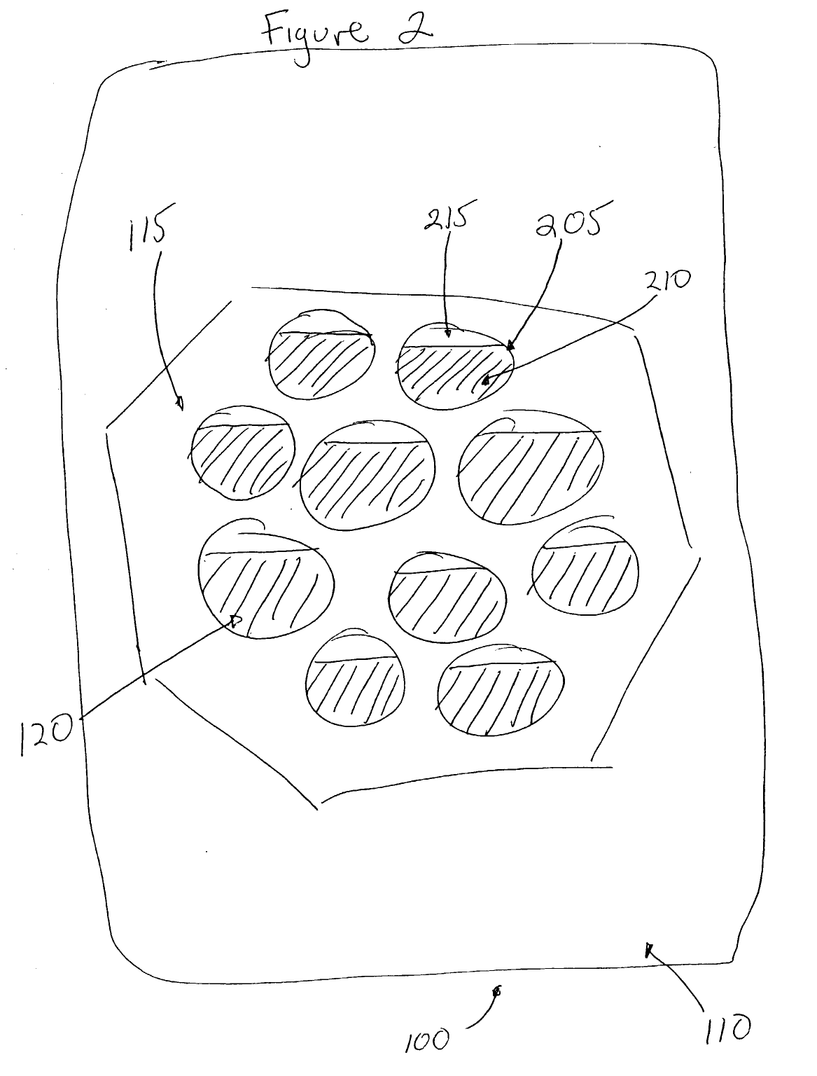

Referring now to FIG. 2, there is seen a cut-away view showing further detail of the cooling modules 120 of FIG. 1. Each of the cooling modules 120 includes a module container 205 and a cooling medium 210 situated within an interior space 215 of the module container 205. The module container 205 may be any shape, for example, substantially spherical, oval, cubic, irregular, etc.

The cooling modules 120 may be dimensioned, for example, to be approximately equal in size and/or to be small relative to the container 110 in which they are contained. In this manner, the cold pack 100 may conform to contours of the area to be subjected to a cold treatment, such as, a portion of a human body, thereby promoting a more effective and uniform application of the cold treatment, as well as reducing the likelihood that the cold pack 100 will slip off the area to be subjected to the cold treatment. Further, since the individually cooling modules 120 may be small relative to the container 110 and move substantially freely to one another, at least some of the cooling modules 120 may come to rest in an area of the cold pack 100 adjacent to at least a portion of the area to be subjected to the cold treatment. In this manner, the cooling modules 120 may individually supply respective pressure points, thereby promoting a more therapeutic effect of the cold pack 100.

It should be noted that, although FIGS. 1-3 show the cooling modules 120 having approximately equal sizes, the cooling modules 120 may be designed, for example, to have varying sizes and/or shapes, which may permit the cold pack 100 to better conform to the contours of the area to be subjected to the cold treatment and/or better promote the therapeutic effect of the cold pack 100.

The module container 205 may include any material operable to contain the cooling medium 210, for example, rigid plastic, flexible plastic, a sponge-like material, a composite material, polyurethane, a non-organic material, a synthetic material, etc.

The cooling medium 210 may include any material operable to provide a cooling temperature to the body. For example, the cooling medium 210 may include a liquid, a solid, or a gelatinous material, for example, liquid water which, when frozen, provides the cooling temperature to the area to be subjected to a cold treatment. In this manner, the cold pack 100 provides the cooling temperature after being externally cooled by a cooling source (not shown), for example, a freezer.

In addition to or in lieu of a liquid, the cooling medium 210 may include, for example, a chemical cooling agent operable to provide the cooling temperature via a chemical reaction, without the need for being externally cooled by the cooling source.

It should be noted that the interior space 215 of the module container 205 may be completely filled with the cooling medium 210 or, alternatively, only be partially filled with the cooling medium 210. Partially filling the module container 205 with the cooling medium 210 may permit the cooling medium 210, for example, liquid water, to expand within the module container 205 while freezing, without rupturing the module container 205.

It should also be noted that, although FIG. 2 shows the module container 205 filled with the cooling medium 210, the interior space 215 of the module container 205 may alternatively be filled with air or be devoid of any cooling medium whatsoever.

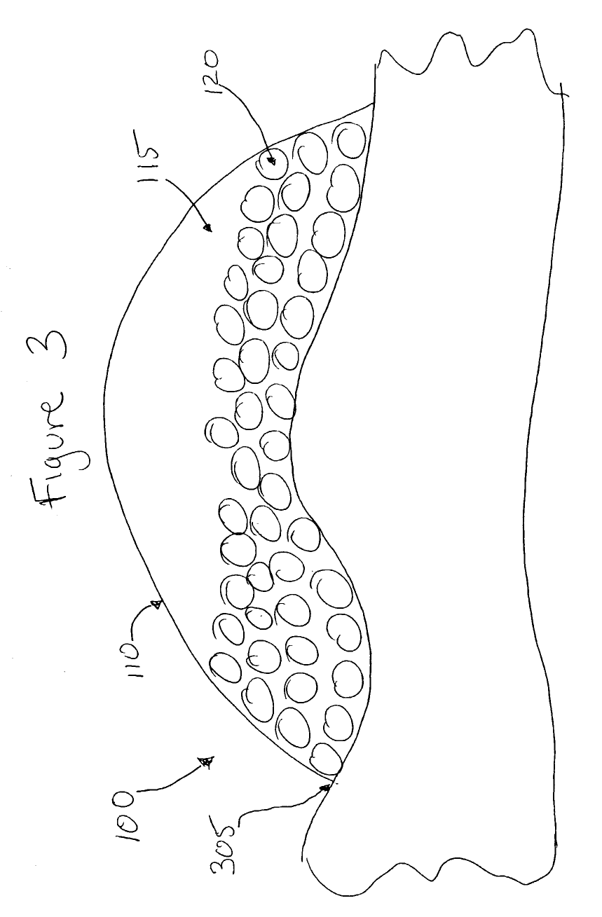

Referring now to FIG. 3, there is seen a diagram showing the application of an exemplary cold pack 100 according to the present invention. When applied to an area to be subjected to a cold treatment 305, for example, an injured portion of the human body (e.g., a human knee), the cooling modules 120 of the cold pack 100 move substantially freely relative to one another, even if the cold pack 100 is applied immediately after being removed from a cooling source, such as a freezer. In this manner, the cooling modules 120 conform to the area to be subjected to a cold treatment 305, without the need for a subdividing deforming force and without the need to wait for the cold pack 100 to at least partially thaw.

Further, it is believed that the ability of the cold pack 100 to conform to the area to be subjected to a cold treatment 305, ensures, or at least makes more probable, that the cold pack 100 will not slip off the area to be treated, for example, the injured portion of the human body.

What is claimed is:

1. A cold pack for providing a cooling temperature to an area to be subjected to a cold treatment, comprising:
a container including an interior space; and
a plurality of cooling modules situated in the interior space of the container, wherein the cooling modules move at least substantially freely relative to one another, without the need for a substantial initial deforming force.

2. The cold pack according to claim 1, wherein the container includes a strong and durable material.

3. The cold pack according to claim 2, wherein the container includes at least one of a rigid material, a semi-rigid material, a flexible material, a malleable material, a conforming material, and a fabric-like material.

4. The cold pack according to claim 3, wherein the container includes at least one of rigid plastic, a flexible plastic, canvas, and polyurethane.

5. The cold pack according to claim 1, wherein the interior space of the container includes a filling medium.

6. The cold pack according to claim 5, wherein the filling medium partially fills the interior space of the container between the plurality of cooling modules.

7. The cold pack according to claim 5, wherein the filling medium completely fills the interior space of the container between the plurality of cooling modules.

8. The cold pack according to claim 5, wherein the filling medium includes at least one of a liquid, air, nitrogen, a gelatinous material, a material resistant to freezing when cooled, Freon, salt-water, anti-freeze, and a chemical that produces the cooling temperature via a chemical reaction.

9. The cold pack according to claim 1, wherein the plurality of cooling modules are small relative to the container and at least some of the plurality of cooling modules are operable to come to rest in an area of the cold pack adjacent to at least a portion of the area to be subjected to the cold treatment, wherein the at least some cooling modules are operable to individually supply respective pressure points to the portion of the area to be subjected to the cold treatment.

10. The cold pack according to claim 1, wherein each of the plurality of cooling modules weighs between about 0.4 grams and 0.6 grams.

11. The cold pack according to claim 1, wherein the cold pack weighs about 454 grams.

12. The cold pack according to claim 1, wherein the container further includes an access apparatus operable to permit access to the interior space of the container.

13. The cold pack according to claim 12, wherein the access apparatus includes at least one of a zipper, velcro, buttons, straps, and snaps.

14. The cold pack according to claim 1, wherein each of the plurality of cooling modules includes a module container having an interior space.

15. The cold pack according to claim 14, wherein the interior space of the module container is one of filled with air and completely devoid of any material.

16. The cold pack according to claim 14, wherein the interior space of the module container is at least partially filled with a cooling medium.

17. The cold pack according to claim 16, wherein the cooling medium includes at least one of a liquid material, a solid material, a gelatinous material, and a chemical cooling agent.

18. The cold pack according to claim 16, wherein the interior space of the module container is completely filled with the cooling medium.

19. The cold pack according to claim 14, wherein the module container includes at least one of a rigid plastic, a flexible plastic, a sponge-like material, a composite material, a non-organic material, a synthetic material, and polyurethane.

20. The cold pack according to claim 14, wherein the module container is substantially spherical in shape.

21. The cold pack according to claim 20, wherein each of the plurality of cooling modules has a diameter of between about 11/32 inches and 13/32 inches.

22. The cold pack according to claim 20, wherein the plurality of cooling modules include 1055 cooling modules.

23. The cold pack according to claim 1, wherein the container includes a substantially rectangular bag sized about 6½ inches by 9 inches and having a thickness of about 1¼ inches when laid flat.

24. A cold pack for treating an injured portion of a human body, comprising:
a container including an interior space; and
a plurality of substantially spherical cooling modules situated in the interior space of the container, the cooling modules being configured to move at least substantially freely relative to one another, even if the cold pack is applied to the injured portion of the human body immediately after being removed from a cooling source.

25. The cold pack according to claim 24, wherein the cooling source includes a freezer.

26. The cold pack according to claim 24, wherein the cooling modules are dimensioned to be approximately equal in size.

27. The cold pack according to claim 24, wherein the cooling modules have at least one of varying sizes and varying shapes.

28. The cold pack according to claim 26, wherein the cooling modules are dimensioned to be small relative to the container.
